(19) United States
(12) Patent Application Publication
(10) Pub. No.: US 2009/0218415 A1
(43) Pub. Date: Sep. 3, 2009

(54) VAPOR-DISPENSING DEVICE COMPRISING AVAILABILITY INDICATOR

(75) Inventor: Geoffrey Faires, Cave Creek, AZ (US)

Correspondence Address: SNELL & WILMER, LLP (Dial Corp), ONE ARIZONA CENTER, 400 E. VAN BUREN, PHOENIZ, AZ, 85004-2202, US

(73) Assignee: THE DIAL CORPORATION, Scottsdale, AZ (US)

(21) Appl. No.: 12/093,194

(22) PCT Filed: Nov. 21, 2006
(86) PCT No.: PCT/US06/45084

§ 371 Date: Jan. 8, 2009

Related U.S. Application Data
(60) Provisional application No. 60/597,358, filed on Nov. 28, 2005.

(51) Int. Cl. A61L 9/04

(52) U.S. Cl. 239/60

(57) ABSTRACT

In general, a vapor-dispensing device (300) includes an indicator system. The indicator system may be configured to automatically indicate the availability status of a vaporizable material within the vapor-dispensing device. The indicator system may comprise a flag or similar object that may be configured to move from a first position to a second position to indicate the availability of the vaporizable material. For example, the availability of a gel type air freshener may be determined from a distance by observing a physical indicator associated with the air freshener. In accordance with another aspect, the indicator system includes two portions (310, 320) of a vapor-dispensing device that are configured to move relative to each other to indicate the availability of the vaporizable material. For example, a gel type vapor-dispensing device may be configured to automatically close when the gel type matrix is substantially exhausted.

### FIELD OF INVENTION

The present invention generally relates to vapor-dispensing devices and, more particularly, to a vapor-dispensing device comprising an availability indicator.

### BACKGROUND OF THE INVENTION

Vapor-dispensing devices are desirable. Vapor-dispensing devices include, for example, air fresheners, insect repellants, and the like. Generally, vapor-dispensing devices contain an exhaustible supply of a volitizable material. However, a user is generally not able to readily notice when the vapor-dispensing device has exhausted its ability to impart the volitizable material to the surrounding environment. For example, to determine if a gel type air freshener has exhausted its fragrance laden gel material, the user of the device typically peers through small holes, slots, or cracks, to see if gel material remains within the body of the device that holds the gel matrix. In some air fresheners, the user disassembles the body to observe the status of the gel matrix.

Thus, there exists a need for a vapor-dispensing device that is configured to clearly indicate when the vaporizable material has been exhausted. For example, a need exists for a gel type air freshener that is configured to clearly signal when the gel matrix has been exhausted. Furthermore, it is desirable to have the ability to observe, from a moderate distance, whether an air freshening device has expended its store of fragrance bearing material. Thus, there is a need for a vapor-dispensing device that overcomes these and other limitations of the prior art.

### SUMMARY OF THE INVENTION

While the way that the present invention addresses the disadvantages of the prior art is discussed in greater detail below, in general, the present invention provides a vapor-dispensing device configured to have an indicator system. The indicator system may be configured to automatically indicate the availability status of a vaporizable material within the vapor-dispensing device.

In accordance with one aspect of the present invention, the indicator system comprises a flag or similar object that may be configured to move from a “full” position to an “empty” position to indicate the availability of the vaporizable material. For example, the availability of a gel type air freshener may be determined from a distance by observing a physical indicator associated with the air freshener. In accordance with another aspect, the indicator system comprises two portions of a vapor-dispensing device that are configured to move relative to each other to indicate the availability of the vaporizable material. In accordance with yet another aspect of the present invention, a gel type vapor-dispensing device is configured to automatically close when the gel type matrix is substantially exhausted.

### BRIEF DESCRIPTION OF THE DRAWINGS

A more complete understanding of the present invention may be derived by referring to the detailed description and claims when considered in connection with the Figures, where like reference numbers refer to similar elements throughout the Figures, and:

FIGS. 1 and 2 are schematic views of exemplary vapor-dispensing devices in accordance with an exemplary embodiment of the present invention;

FIG. 3 is another exemplary vapor-dispensing device in accordance with an exemplary embodiment of the present invention;

FIGS. 4-6 are cross-sectional views of exemplary vapor-dispensing devices in accordance with various embodiments of the present invention; and

FIG. 7 is a perspective, partial cut-away view of an exemplary vapor-dispensing device in accordance with various exemplary embodiments of the present invention.

### DETAILED DESCRIPTION

The following description is of exemplary embodiments of the invention only, and is not intended to limit the scope, applicability or configuration of the invention in any way. Rather, the following description is intended to provide a convenient illustration for implementing various embodiments of the invention. As will become apparent, various changes may be made in the function and arrangement of the elements described in these embodiments without departing from the scope of the invention as set forth in the appended claims. For example, in the context of the present invention, the method and apparatus hereof find particular use in connection with air freshening systems. However, generally speaking, various vapor-dispensing devices that are suitably configured to emit various fragrance materials such as deodorizers, sanitizers, insecticides, insect repellants, and/or the like are suitable for use in accordance with the present invention. Likewise, though various portions of the specification refer to a gel matrix as the fragrance material used herein, various other vaporizable materials now known or as yet unknown, having similar properties should be considered within the scope of the present invention.

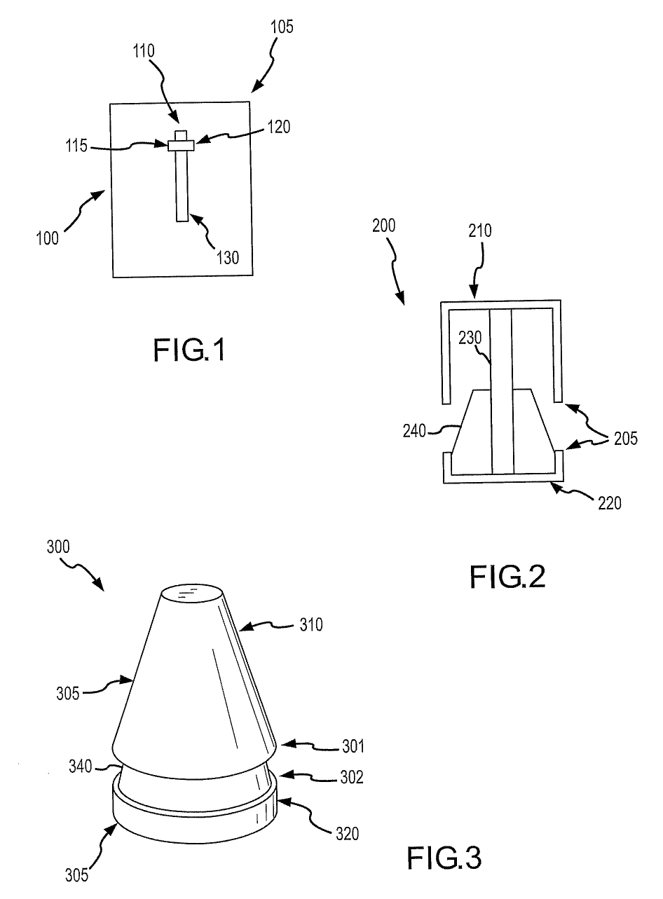

With reference now to FIG. 1, a vapor-dispensing device 100 is configured to indicate the availability of a vaporizable material. In accordance with one exemplary embodiment of the present invention, vapor-dispensing device 100 comprises a vaporizable material (not shown), a housing 105, and an indicator system 110. Indicator system 110, as described in greater detail herein, may be configured to indicate the availability of the vaporizable material. The availability of the vaporizable material may be indicated, for example, by the movement of an object 115 from a “full” position 120 to an “empty” position 130.

By way of example, vapor-dispensing device 100 may be an air freshener device comprising an air freshening material, such as a gel matrix, and the air freshener may be suitably configured to indicate the availability of the gel matrix. Thus, vapor-dispensing device 100 may be configured such that a consumer may readily observe whether it is time to purchase a refill for or a replacement of vapor-dispensing device.

Briefly, the vaporizable material may comprise a material that can be vaporized over a period of time. In exemplary embodiments, the vaporizable material comprises a fragrance, an insect repellant, a deodorizer, a sanitizer, an insecticide, and/or the like. In one exemplary embodiment, the vaporizable material comprises an air freshening material.

The vaporizable material may also comprise a support structure that is configured to temporarily hold the material that can be vaporized. For example, the support structure may be a gel matrix. In this regard, the gel matrix may be any conventional gel or gel type product, now known or as yet unknown. Furthermore, other suitable gel matrix substances may be used in various embodiments of the present invention. Moreover, the support structure may be a liquid, an oil, and/or other material providing a support structure that is suitably configured to hold and/or deliver a vaporizable substance (e.g., a fragrance).

The support structure is further configured to change as the fragrance is exhausted. For example, the support structure (e.g., the substance bearing the fragrance) may be configured to physically change size as the fragrance is communicated to the surrounding environment. In one exemplary embodiment, as fragrance material is delivered from a gel matrix, the gel matrix may contract. Stated another way, the volume of the volitizable material may reduce as the volitizable material is communicated to surrounding environment. In yet another exemplary embodiment, as fragrance material is delivered from an oil reservoir, the volume of the fragrance laden oil is reduced.

Thus, over a period of time, the volitizable material may be depleted from the vapor-dispensing device. The vapor-dispensing device may begin with a fall capacity of the volitizable material and end with substantially no volitizable material (i.e., less than 5% of the volitizable material left). Once a vapor-dispensing device has substantially no volitizable material left to dispense, it may be said that the material has been exhausted. However, it has previously not been as easy to determine whether the volitizable material had become exhausted without peering through small holes or slots in the housing to investigate the availability status of the volitizable material, as it is with the various embodiments of the present invention.

In accordance with one exemplary embodiment of the present invention, housing 105 is configured to support the volitizable material. This support may comprise no more than, for example, a gel matrix being supported on its bottom side and/or about an interior column about which the gel matrix is formed. In other embodiments, the volitizable material may be supported on the bottom, sides, and/or top, as, for example, in case of a vial of oil having a wick inserted therein. In general, housing 105 may be made of any suitable material. In one exemplary embodiment, housing 105 is constructed of a plastic material.

Housing 105 may comprise holes, slots, vents, and other configurations commonly used for allowing the vapor to be transmitted from the housing to the surrounding environment. Furthermore, the system for transmitting the vaporized material to the surrounding environment may be configured to control the release of the material. For example, the rate of the release may be controlled by adjusting the size of the holes, the size of the opening, the percentage of the holes that are open, and/or the like. Furthermore, the housing may be configured to have an open and a closed position. In the open position, the fragrance may be dispersed to the surrounding environment, and in the closed position, the fragrance may be retained within the housing during initial shipping/sale of the product, and during periods of non-use, and/or storage.

In one exemplary embodiment, housing 105 may be suitably configured to resemble typical air freshener housings, insect repellant housings, deodorizer housings, and/or the like that are now available, previously available, or here after developed. For example, with reference to FIG. 3, housing 305 may be a semi-conical shaped gel type air freshening device, though housing 105 may comprise any suitable shape for containing and facilitating the controlled release of a vaporizable material.

In accordance with one aspect of the present invention, vapor-dispensing device 100 comprises an object 115 that moves relative to housing 105. With reference now to FIG. 2, and in accordance with another aspect of the present invention, housing 205 may further comprise a first portion 210 and a second portion 220 that are configured to move relative to each other. For example, the first portion may comprise an upper portion or cap (e.g. 210, 310), and the second portion may comprise a lower portion or base (e.g., 220, 320). In this example, vapor-dispensing device 300 may be open when edges 301 and 302 are apart and closed when the edges are brought together.

Vapor-dispensing device 200 may further comprise an intermediate portion 230. Intermediate portion 230 may be configured to facilitate relative motion between lower portion 210 and upper portion 220. For example, intermediate portion 230 may comprise concentric sliding tubes, a screw mechanism, and or the like.

In accordance with one embodiment, the vapor-dispensing device is configured such that an individual observing the outside surface of the air freshener/vapor-dispensing device is able to determine the amount of the vaporizable material inside the housing. For example, the height of a gel matrix inside an air freshener may be readily observed by looking at the outside of the device. The individual is able to do so without looking inside the housing or without looking at the volitizable material (e.g., the gel matrix) itself.

In accordance with one exemplary embodiment, the vapor-dispensing device is configured to have an indicator object that moves relative to the housing. The indicator object may move from a first position, representing a fully charged product, to a second position representing a depleted product having substantially no vaporizable material remaining. With reference again to FIG. 1, indicator system 110 may comprise an indicator object 115 that moves relative to the availability of the vaporizable material. For example, indicator object 115 may comprise a physical element such as a flag, a bar, and/or the like. Indicator object 115 may be positioned at a first position 120 illustrating to the observer that the vapor-dispensing device is fully loaded. The indicator object may be configured to move to a second position 130, which illustrates that the vapor-dispensing device has been depleted of the vaporizable material.

In this regard, the housing may be suitably configured with markings, labels, stickers, or other devices for illustrating the significance of the position of the indicator object. For example, the first position 120 of indicator object 115 may be marked with the words “full”, “fully charged”, “new”, and/or the like. The second position 130 of indicator object 115 may be marked with the words “empty”, “exhausted”, “replace with a new unit”, and/or the like. Furthermore, symbols, colors or other devices may be used to convey similar messages.

Indicator system may be configured to move in approximately an analog manner, i.e., approximately proportionate to the depletion of the gel like material. In yet another embodiment, the indicator system is configured to move in a binary manner, in that the indicator object may remain substantially at its first “available” position until the vaporizable material is nearly exhausted, and then move to the second “unavailable” position.

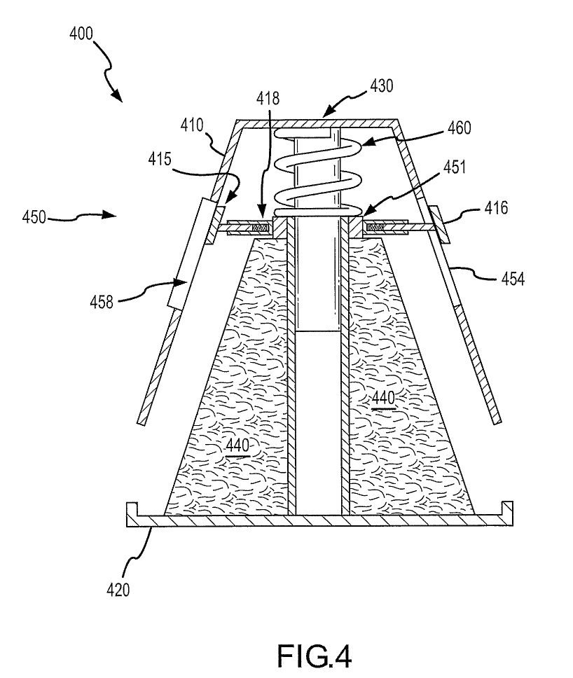

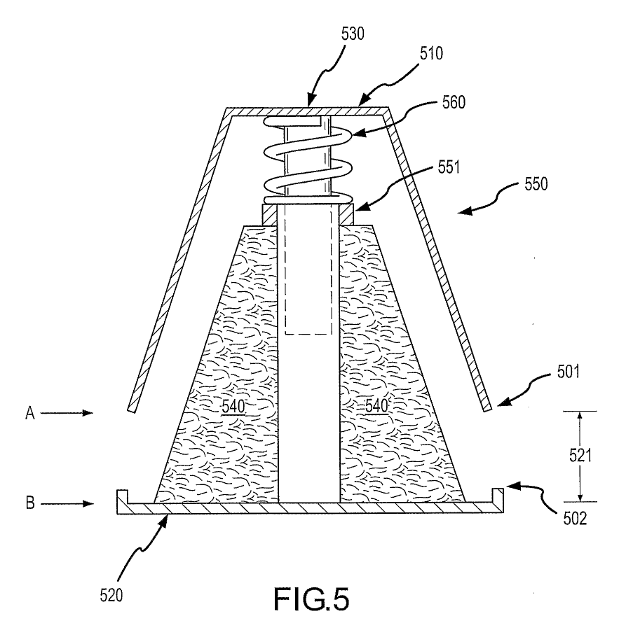

Indicator object 115 may be configured in a number of ways to move in accordance with the availability of the vaporizable material. For example, with reference to an exemplary gel type vapor dispenser and FIG. 4, a typical gel type air freshener 400 is configured with an intermediate portion 430 that allows a cap 410 to be lifted and suspended above a base 420 in order to open the air freshener. Air freshener 400 also comprises a gel matrix 440. Vapor-dispensing device 400 further comprises an indicator system 450. Indicator system 450 may further comprise a level ring 451 riding on top of gel matrix 440. In one embodiment, level ring 451 is the indicator object. In another embodiment, level ring 451 may comprise one or more indicator object(s) (e.g., 415, 416) attached thereto.

In one exemplary embodiment, indicator object(s) 415 are positioned inside cap 410. In this embodiment, cap 410 may be configured to have a window or slot 453 through which indicator objects may be viewed. In yet another embodiment, level ring 451 and/or indicator object(s) 415 may comprise a magnetically polarized portion, and cap 410 may be configured with a magnetically sensitive tape/window. An exemplary magnetically sensitive tape/window (also referenced as 453), may be configured to reveal or display the height of gel matrix 440.

In another exemplary embodiment, indicator object(s) 416 may be positioned outside cap 410. In this embodiment, cap 410 may comprise a slot 454 through which indicator objects 416 may be attached to level ring 451. Thus, indicator object(s) 416 may be displayed on the outer surface of housing cap 410.

Whether indicator object 415 or 416 are inside or outside of cap 410, they may further be configured to remain in close proximity to cap 410. For example, the indicator object(s) (e.g., 415, 416) may be configured to slide in and/or out in a horizontally radial direction from the axis of level ring 451. Furthermore, indicator object(s) (e.g., 415, 416) may be biased towards cap 410 via, for example, springs 418, hydraulics, magnetic force, mechanical guides on cap 410, and/or the like.

Indicator object(s) (e.g., 415, 416) may be configured to move under the influence of gravity. In other words, the indicator object(s) move with the level ring which is maintained in contact with the upper surface of gel matrix 440 by gravity. In this embodiment, the level ring is held to the top of gel matrix 440 by gravity and follows the top of gel matrix 440 as it contracts downward when vaporizable material is dispensed. Indicator object(s) (e.g., 415, 416) may, however be influenced to move under the force of springs, hydraulic pressure, elastic, rubber-bands, and/or the like. For example, level ring 451 may be biased downward by spring 460.

In yet another exemplary embodiment of the present invention, the indicator system may comprise components of the housing itself. For example, housing 305 may be configured to have at least two components, and the relative movement of the two components may be configured to indicate the availability of vaporizable material 340. In accordance with an exemplary embodiment of the present invention, housing 305 may comprise a housing cap 310 and a housing base 320. Housing cap 310 is configured to move relative to housing base 320 such that the relative movement indicates the availability status of vaporizable material 340. For example, the relative separation of cap 310 and base 320 may indicate the availability of vaporizable material 340.

The indicator system may be configured in various ways such that a change in the availability of the fragrance material changes the relative separation of two portions of the housing. In one exemplary embodiment, and with reference to FIG. 5, indicator system 550 comprises a spring 560 attached to level ring 551 on one end and to cap 510 on the other end of spring 560. Level ring 551 is, for example, an annular ring. Level ring 551 may be disposed about an intermediate portion 530, which connects cap 510 to base 520. Intermediate portion 530 may comprise, for example, coaxial right cylindrical tubes with an inner portion configured to slide within an outer portion.

Indicator system 550 may be configured such that spring 560 is in a state of compression while cap 510 is in the closed position (full matrix). Although less compressed, spring 560 may also be compressed when cap 510 is raised to an open position (full matrix), as illustrated in FIG. 5. Cap 510 is thus supported in an open position by spring 560. Cap 510 is restrained from further upward movement by, for example, interference between the inner and outer portions of intermediate portion 530.

As gel matrix 540 begins to contract, level ring 551 is biased away from cap 510 and spring 560 extends. Indicator system 550 is configured such that when the level ring reaches a Point A, farther contraction of matrix 540 causes cap 510 to follow the matrix down. For example, at Point A spring 560 may be fully extended or may be restrained from farther extension. When matrix 540 is exhausted, level ring 551 may be at a Point B. The distance between Point A and Point B may, for example, be the same distance as the distance between the two housing portions 510 and 520 when fully open (e.g., the distance 521 between edges 501 and 502). Thus, when matrix 540 is substantially exhausted, cap 510 is closed to the base. Therefore, vapor-dispensing device 500 may be configured, in various manners, to indicate that the vaporizable material is no longer available by automatically closing the vaporizable dispensing device.

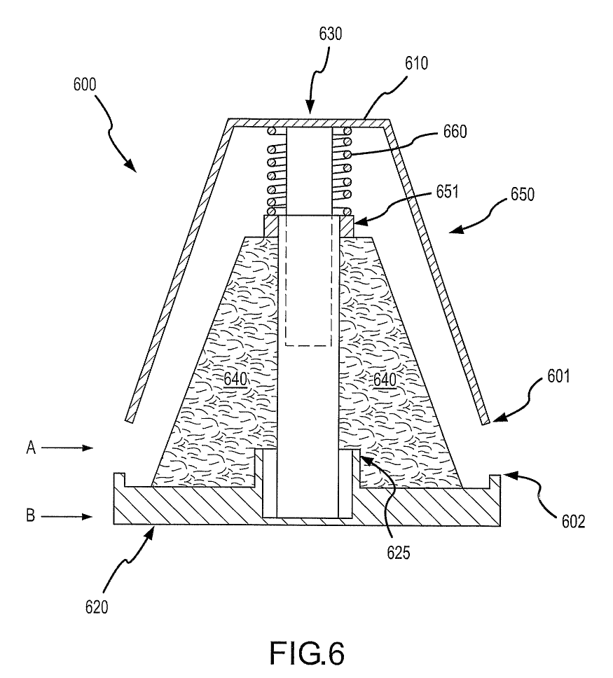

In yet another version of this exemplary embodiment, and with reference to FIG. 6, vapor-dispensing device 600 may be configured to close in a binary on/off manner. For example, device 600 may be configured to close when a substantial amount of the volitizable material has been exhausted, e.g. 90% or more. In this exemplary embodiment, base 620 may comprise a level ring receiving portion 625 configured to receive level ring 651. Level ring receiving portion 625 may be configured to be as deep as the separation between edges 601 and 602.

In this example, Point A, where the change in the height of level ring 651 also changes the height of cap 610, may be located at the top of recessed portion 625. When volitizable material 640 has contracted to the point that level ring 651 is at Point A, substantially all of the volitizable material is exhausted. From Point A, level ring 651 may fall into level ring receiving 625. Thus, vapor-dispensing device 600 may be configured such that the change in position of level ring 651 from Point A to a Point B, at or near the bottom of level ring receiving portion 625, causes cap 610 to close.

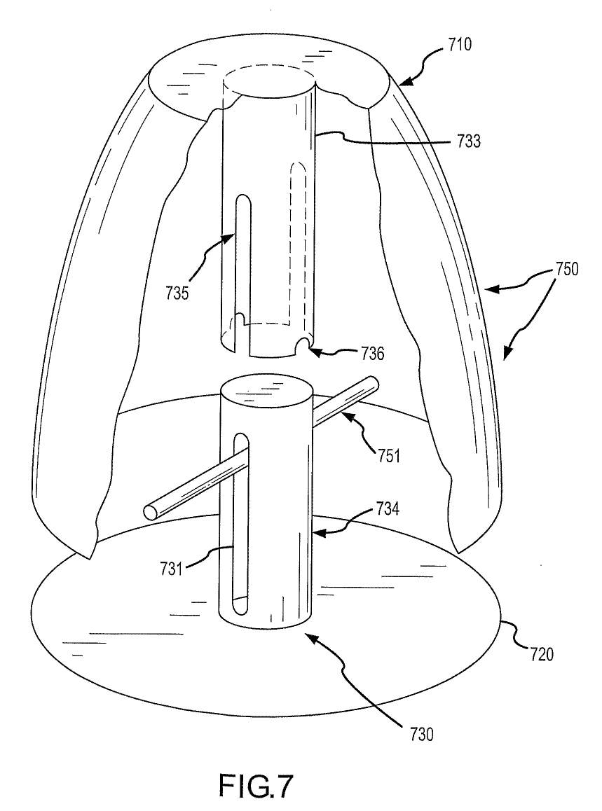

In yet another exemplary embodiment, and with reference to FIG. 7, indicator system 750 is configured such that the interior of the gel matrix serves to hold the cap in the open position. In this embodiment, a level bar 751 is configured to support cap 710. Level bar 751 may comprise, for example, a horizontal member configured to pass perpendicularly through a slot 731 in intermediate portion 730. Level bar 751 may be configured to slide up and down in slot 731 and may be supported in a raised position 732 by the gel matrix, which surrounds the portions of level bar 751 that extend outside of intermediate portion 730. Thus, the initial reduction of volume in the gel matrix has little effect on the position of level bar 751. However, as the gel matrix contracts below the initial position of level bar 751, level bar 751 may begin to descend as well. In this exemplary embodiment, intermediate portion 730 is further configured such that a change in position of level bar 751 results in a change in position of cap 710.

For example, intermediate portion 730 may further comprise an inner portion 733 and an outer portion 734. Inner portion 733 may be configured to slide coaxially within outer portion 734. In one exemplary embodiment, inner portion 733 and outer portion 734 comprise right cylindrical tubes. Furthermore, inner portion 733 may be connected to cap 710 such that as inner portion 733 moves up or down inside outer portion 734, cap 710 moves up or down accordingly. Inner portion 733 may also be configured to have a major slot 735 that allows inner portion 733 to slide down over level bar 751. Major slot 735 may be configured to have a length sufficient to facilitate closing cap 710 to base 720 when level bar 751 is at its initial height. Inner portion 733 may also be configured to have a minor slot or retention slot 736. In an exemplary embodiment, minor slot 736 is 90 degrees from major slot 735.

Inner portion 733 is therefore configured to be raised (with cap 710), turned 90 degrees, and supported by level bar 751. For example, inner portion 733 may be supported by placing level bar 751 securely in minor slot 736. As level bar 751 eventually lowers, cap 710 may also lower with the bar. Furthermore, in the event that a user wishes to temporarily close vapor-dispensing device 700, cap 710 may be lifted, rotated 90 degrees, and lowered to the closed position.

In various embodiments, the vapor-dispensing device may be further configured such that the cap and the base may be ‘locked’ or held together. The vapor-dispensing device may, for example comprise a connection mechanism that is configured to secure the cap to the base when the device is in a closed position. The connection mechanism may comprise a snap, clip, twist and lock, and/or other similar junction. In various examples, the device may be opened and closed multiple times.

In general, the indicator system may configured to indicate the availability status of a vaporizable material through relative movement of two objects, The first object may be a flag that moves relative to a housing, and/or the first object may be one portion of the housing that moves relative to a second portion of the housing. In either event, the relative movement discloses to an observer the availability status. In various embodiments, the indicator system is configured to automatically close the housing when the vaporizable material 240 is nearly exhausted.

The present invention has been described above with reference to various exemplary embodiments. However, many changes, combinations and modifications may be made to the exemplary embodiments without departing from the scope of the present invention. For example, the various components may be implemented in alternate ways. These alternatives can be suitably selected depending upon the particular application or in consideration of any number of factors associated with the operation of the system. In addition, the techniques described herein may be extended or modified for use with other types of devices. These and other changes or modifications are intended to be included within the scope of the present invention.

We claim:

1. An air freshener comprising:
a gel matrix;
a housing configured to support said gel matrix; and
an indicator system configured to indicate the availability status of said gel matrix; wherein said indicator system comprises a first component and a second component that are configured to move relative to each other based on the reduction in volume of said gel matrix, and wherein the availability status of said gel matrix is illustrated by said relative movement of said first and second components.

2. The air freshener of claim 1, wherein said first component and said second component are configured to move relative to each other such that said first and second components may be moved from a first position to a second position, said first position representing an open position and said second position representing a closed position; and wherein said first and second components are configured to automatically move from said open position to said closed position when said gel matrix is exhausted.

3. The air freshener of claim 2, wherein said first portion moves relative to said second portion under the influence of gravity.

4. A self-closing air freshener, wherein said self-closing air freshener comprises:
a gel matrix;
a first portion;
a second portion, wherein at least one of said first and second portions is configured to support said gel matrix, and wherein said first and second portions are configured to move relative to each other; and
an adjustment portion, wherein said adjustment portion is configured to facilitate the movement of said first portion relative to said second portion from a first position to a second position based on the contraction of said gel matrix.

5. The self-closing air freshener of claim 4, wherein said first and second portions move from an open position to a closed position based on the availability of said gel matrix.

6. The self-closing air freshener of claim 4, wherein the relative movement of said first and second portions occurs under the influence of gravity as said gel matrix contracts.

7. The self-closing air freshener of claim 5, wherein said air freshener is configured to close when said gel matrix is substantially exhausted
